(19) United States
(12) Patent Application Publication
(10) Pub. No.: US 2019/0175820 A1
(43) Pub. Date: Jun. 13, 2019

(54) A DELIVERY SYSTEM INCLUDING A POSITION DETECTING UNIT

(71) Applicant: MEDTRUM TECHNOLOGIES, INC., Shanghai (CN)

(72) Inventor: Cuijun YANG, Shanghai (CN)

(21) Appl. No.: 16/321,927

(22) PCT Filed: Aug. 12, 2016
(86) PCT No.: PCT/CN2016/094957

§ 371 Date: Jan. 30, 2019

(51) Int. Cl. A61M 5/145; A61M 5/168

(52) U.S. Cl. CPC A61M 5/1456; A61M 2205/3317; A61M 5/16831

(57) ABSTRACT

Disclosed is a delivery system including a position detecting unit, comprising a housing (1), a reservoir (2), a piston (3), a lead screw (4) and at least two or more position sensors (5a,5b,5c,5d,5e), wherein the piston (3) includes a detectable feature. The position sensors (5a,5b,5c,5d,5e) are arranged on or adjacent to the outer wall of the reservoir (2) composing a sensor array, configured to sense a change of the magnetic field or the electric field caused by the displacement of the detectable feature to generate a signal based on which the position of the piston (3) can be determined using algorithms, and to detect the infusion abnormality by sensing the displacement abnormality of the piston (3), endowing an infusion device with performances as compact structure, low cost, high precision, fast reaction and a minimum volume under the premise of safety guarantee.

### TECHNICAL FIELD

The present invention generally relates to the field of medical appliance, more particularly, to a delivery system including a position detecting unit.

### BACKGROUND OF THE INVENTION

An ambulatory infusion pump device is always used by a patient with a continuous and in the course of the day, varying need of a fluid infusion. Such devices are particularly useful for ambulatory therapy and are generally carried attached on the body of a patient. The reservoir often comprises fluid supply sufficient for one or several days. The fluid is supplied to the patient's body from the reservoir through an infusion cannula or an injection needle.

Ambulatory infusion pump devices are typically of the syringe driving type. A number of drawbacks of such syringe-type pump designs are known in the prior art. In particular, such pump devices have a limited precision, because very small volumes, typically in the nanoliter range, are pumped out of a reservoir with an overall volume in the range of milliliters. To achieve precise dosing, it is necessary to displace the piston very precisely. Even small deviations can lead to over dosing or under dosing, and the forces needed to actuate the piston are comparably high due to the friction between the walls of the reservoir and the seals of the piston and the material hysteresis of the seals. This leads to demanding requirements for the drive system and the mechanical parts involved, as well as the control unit of the pump. As a consequence, such infusion pump devices are expensive.

Another problem is the lower limit of the length of such an infusion pump device. The complete supply of fluid has to be stored in the reservoir, and the cross-sectional area of the piston has to be below a certain limit, for precision reasons and in order to limit the device thickness, which is known to be a particularly critical dimension with respect to comfort and discreetness during application. Thus, the minimum overall length of the device is then essentially given by the resulting minimum length of the reservoir, which is detrimental to the provision of compact infusion pumps. The traditional infusion pump usually has a plunger which is used to pump out the fluid stored in the reservoir with a plunger head and a plunger shaft, and a drive motor is further arranged to drive the plunger shaft, and this combination of design compromises the portability of the infusion pump with multiple components, complex structures and large volume.

In an alternative approach, a separate dosing unit is provided downstream from the reservoir. Since the primary reservoir does not have to fulfill additional functions, its dimensions can be optimized in view of the compactness of the infusion pump device. Such a dosing unit may for example comprise a micro piston pump with small dimensions that retrieves fluid from a larger primary reservoir and conveys the fluid to the body of the patient. Such pumps are generally full-stroke pumps, where the cavity of a membrane pump or the cylinder of a piston pump is always completely emptied. Hence, the inner volume of the pump must correspond to the smallest volume increment to be delivered, typically in the nanoliter range. While several designs for such dosing units are known in the prior art, they are rather complex, expensive and critical with respect to large scale manufacture, and inconvenient to use.

Regardless of how the delivery system is designed, for precise metering, it is necessary to either use a pump motor that can be very precisely controlled, for example a stepper motor, or to monitor the actual position of the piston. US2014039396A1 disclosed a dosing unit with an optical position detecting system. It has a cylinder pump comprising a cylinder and a piston which comprises a piston head and a piston shaft which has a segment provided with markings that can be optically detected, and the markings are a plurality of stripes arranged on the shaft segment that can be interacted with an optical sensor to detect the position of the piston; and US20080077081A1 disclosed an infusion pump device with a cylinder pump having a longitudinally displaceable piston with a split piston shaft that connects the piston head with a threaded nut. The threaded nut interacts with a rotating threaded drive shaft, thereby transferring the rotation of the drive shaft into a linear displacement of the piston head. One or more detectable features such as magnetic or optical markers can be arranged on the piston shaft, which can be detected by a corresponding sensor for determining the linear position of the marker, and thus of the piston head. However, the two designs mentioned above still have the disadvantages of multiple components, large volumes and restricted precision. More importantly, if the magnetic sensor is applied as the position sensor, its precision can be compromised by interferences from a variety of environmental factors. The farther away from the detectable feature the magnetic sensor is, the more harmful interference the detecting system will receive, and the poorer the detecting precision will be. Additionally, unlike a sensor array, using only one position sensor cannot realize segmented detection whose precision was ensured by always having one sensor within a sufficiently short distance to the detectable feature.

US2014197824A1 disclosed an infusion pump including a syringe plunger position sensor array. A linkage with a syringe plunger engagement member is arranged in the housing of the pump device, a magnet is set on the linkage and the sensor array is set on the arm of the linkage, configured to determine the linear position of the syringe plunger using a software based on an output generated by the sensor array indicative of a magnetic field associated with the magnet. However, the magnet used as the detectable feature in the disclosure identified above is still arranged on an additional member connected with the plunger, and the existence of this additional member seriously compromises the compactness of the infusion device which is crucial to the portability, one of the key performances, of this kind of device. Furthermore, the displacement of the engagement member does not equal directly to the displacement of the plunger, making the test results bearing unneglectable errors.

### SUMMARY OF THE INVENTION

To overcome shortcomings in the prior art mentioned above, the purpose of the present invention is to provide a delivery system including a position detecting unit, comprising:

a housing;

a reservoir supported by the housing;

a piston displaceable along the longitudinal axis in the reservoir, wherein the piston includes a detectable feature;

a lead screw directly connected with the piston;

at least two or more position sensors, wherein the position sensors are arranged within the longitudinal distance of the reservoir composing a sensor array, configured to sense a change of the magnetic field or the electric field caused by the displacement of the detectable feature and generate a signal. Based on the signal the position of the piston can be determined using algorithms, and any displacement abnormalities of the piston can be sensed from which infusion abnormalities can be deduced.

Alternatively, the sensor array is a linear array of sensors, with every sensor detecting the displacement of the piston for a specific range, configured to realize segmented detections of the detectable feature;

Alternatively, the position sensors are arranged on the outer wall of the reservoir in the same housing with the reservoir;

Alternatively, the position sensors are arranged adjacent to the reservoir in the other housing which can be connected with the housing supporting the reservoir;

Alternatively, the number of the position sensors in the linear sensor array is 5 to 10, and the position sensors can realize joint detections with a specific algorithm.

Alternatively, the position sensors are magnetic sensors; Alternatively, the detectable feature is the piston itself made of magnetic plastic;

Alternatively, the detectable feature is a magnet foil which is mounted in a recess set on the piston;

Alternatively, the detectable feature is a magnetic silicone sealing ring set in the circumferential direction on the piston;

Alternatively, the detectable feature is a magnetic rubber sealing ring set in the circumferential direction on the piston.

Alternatively, the detectable feature is a metal key which is mounted in a recess set on the piston;

Alternatively, the position sensors are capacitive sensors;

Alternatively, the position sensors are inductive sensors;

Alternatively, the position sensors are eddy-current sensors.

The delivery system including a position detecting unit provided by the present invention has a compact piston with a very short longitudinal length, no volume-consuming shaft attached, nor volume-consuming drive motor to drive the shaft. A lead screw is directly connected with the compact piston and drives its displacement along the longitudinal axis in the reservoir. The detectable feature is directly set on the piston or is the piston itself, whose displacement change leads to a magnetic or electric field change, detected by the position sensor to generate a real-time displacement signal by which a real-time amount of fluid administrated from the reservoir can be calculated, in order to achieve the purpose of precise control of the infusion status and detecting abnormalities such as blockage and lead screw breakage, etc. The most obvious advantage of this arrangement of the detection method lies in that no additional member linked with the piston is needed to hold the detectable feature or sensors, meaning no compromise of compactness of the pump device which is critical to the key performance of portability needs to be made, and errors caused by the possible inequality between the displacement of the engagement member and the displacement of the plunger can be avoided. There are at least two or more position sensors, which can be arranged either side by side with the reservoir within the housing, or directly on the outer wall of the reservoir, to make sure that they are close enough to the detectable feature. In some optimized embodiments, multiple sensors compose a sensor array to further realize segmented detections within a restricted distance. For each segment, only the sensor closest to the detectable feature among all sensors is used as the primary one to determine the displacement, which minimizes the harmful interference and ensures the detection precision to a maximum degree. Additionally, the position sensors can realize joint detections with a specific algorithm. To sum up, The delivery system including a position detecting unit in the present invention has advantages as compact structure, low cost, high precision, fast reaction, achieving a minimum volume under the premise of safety guarantee and a very high precision of detection by careful arrangement of the detecting units, which reflects the combination of the two major advantages of portability and safety of the portable infusion device.

### BRIEF DESCRIPTION OF THE DRAWINGS

FIG. 1 is a schematic view of an embodiment of the present invention

FIG. 2 is a perspective of an embodiment of the present invention

FIG. 3 is a schematic view of another embodiment of the present invention

FIG. 4 is a perspective of a piston in a reservoir of the present invention

FIG. 5 is a schematic view of another embodiment of the present invention

### DETAILED DESCRIPTION

To make the above-mentioned objects, features and advantages of the present invention more obvious and understandable, the embodiments of the present invention are described in the following through specific embodiments.

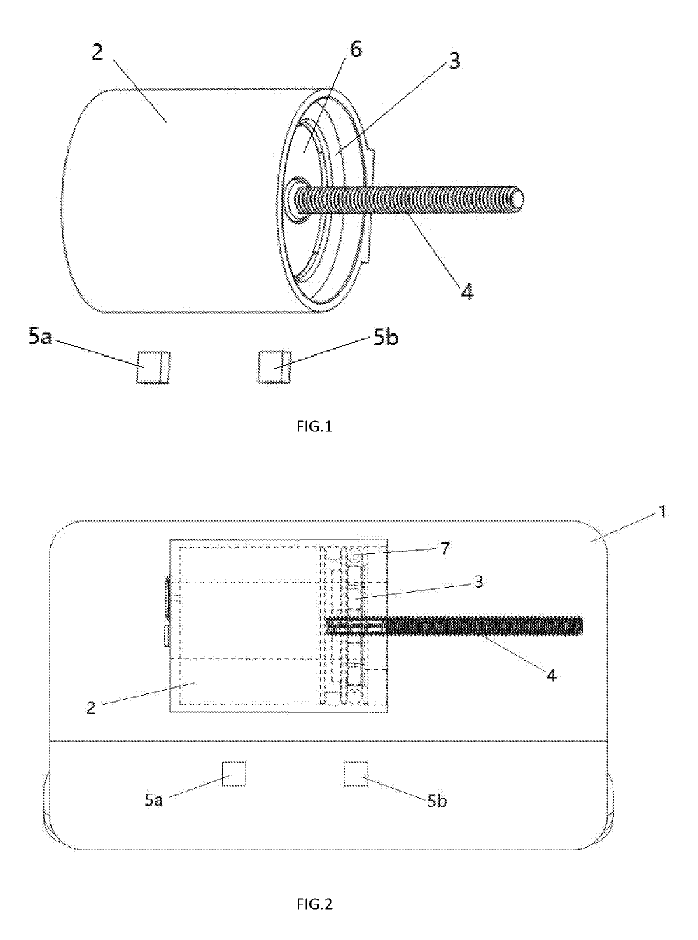

Referring to FIG. 1 and FIG. 2, an embodiment of the delivery system including a position detecting unit in the present invention is provided. A reservoir 2, a piston 3 and a lead screw 4 are arranged in a housing 1, and two position sensors 5a and 5b composing a linear sensor array are arranged in the other housing connected with the housing 1 within the longitudinal distance of the reservoir, and position sensors 5a and 5b are magnetic sensors. The piston 3 can displace inside the reservoir 2 along the longitudinal axis. A magnet foil 6 with the same shape as the cross-section of the piston 3 is mounted in a recess set on the piston 3 as a detectable feature. The lead screw 4 is connected to the piston 3 directly to drive the piston 3 moving forward in the reservoir 2. The two position sensors 5a and 5b are arranged adjacent to the linear path of the displacement of the piston 3, each detecting the displacement of the piston 3 for a primary range. The magnet foil 6 has a magnetic field associated therewith. Each position sensor can sense the changes of the magnetic field based on which the actual position of the piston 3 can be determined using specific algorithms without initial calibration. Within a specific range, the sensor 5a is used as the primary sensor to generate a primary signal, but the sensor 5b can also function as a secondary sensor to generate a secondary signal which can also be used in determining the position of the piston 3, realizing both the segmented detection based on applying the signal provided by the primary sensor in priority within a specific range and the joint detection based on the cooperation of multiple sensors, which make the detection result more reliable and accurate.

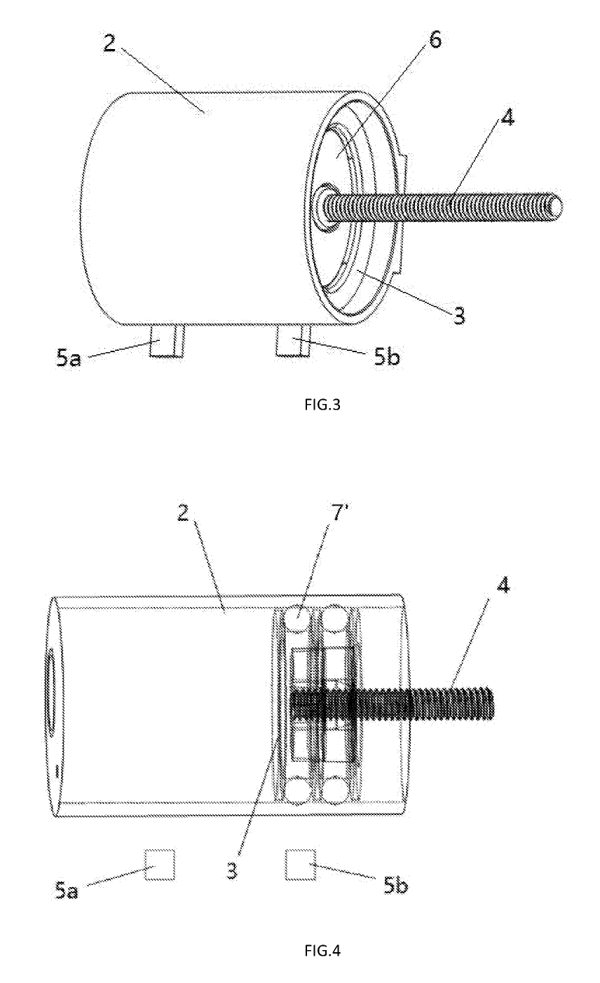

Referring to FIG. 3, another embodiment of the delivery system including a position detecting unit in the present invention is provided. In this embodiment, the sensors 5a and 5b are arranged directly on the outer wall of the reservoir 2 in the same housing with the reservoir. In this arrangement, the position sensors are closer to the magnetic foil 6 as the detectable feature, making the magnetic field less subject to interferences such as the geomagnetic field, further enhancing the accuracy of the detecting results.

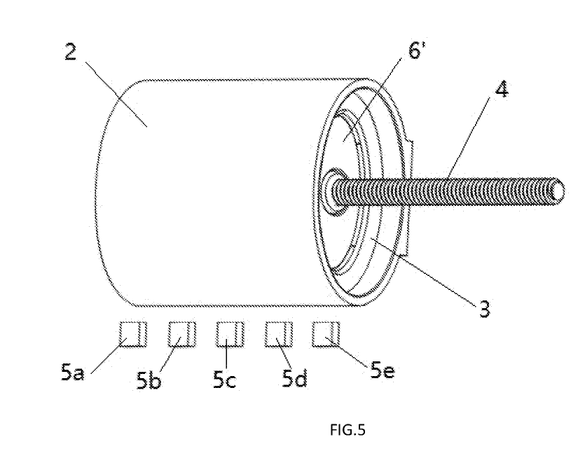

Referring to FIG. 2 and FIG. 4, when the position sensors in the sensor array remain to be magnetic sensors, there are other alternatives for the detectable feature other than a magnet. For instance, the detectable feature can be the piston 3 itself made of magnetic material, or a separable member combined with the piston as a magnetic silicone or rubber ring set in the circumferential direction on the piston 3. In one embodiment, the detectable feature is a magnetic silicone sealing ring 7 as shown in FIG. 2; in other embodiment, the detectable feature is a magnetic rubber sealing ring 7′ as shown in FIG. 4. As the same function with the magnetic foil 6 in above-mentioned embodiments as a detectable feature, the magnetic piston 3, magnetic silicone sealing ring 7 or magnetic rubber sealing ring 7′ has a magnetic field associated therewith. Sensors 5a and 5b sense the changes of the magnetic field based on which the position of the piston 3 can be determined using specific algorithms.

Referring to FIG. 5, another embodiment of the delivery system including a position detecting unit in the present invention is provided. In this embodiment, the detectable feature is a metal key 6′, and the position sensors are capacitive sensors, and there are five capacitive sensors 5a, 5b, 5c, 5d and 5e composing a sensor array, each detecting the displacement of the piston 3 for a primary range. The metal key 6′ has an electric field associated therewith. Each sensor can sense the changes of the electric field based on which the position of the piston 3 can be determined via specific algorithms without initial calibration. Within a specific range, the sensor 5a is used as the primary sensor to generate a primary signal, but the sensors 5b and 5c can also function as secondary sensors to generate secondary signals which can also be used in determining the position of the piston 3, realizing a joint detection less subject to interferences from public electric field, which makes the detection result more reliable and accurate. Similarly, when sensor 5d is used as the primary sensor, sensors 5c and 5e can be used as secondary sensors to realize a joint detection. In this embodiment, the position sensors are capacitive sensors, in other embodiment, the position sensors can be inductive sensors or eddy-current sensors, with the metal key 6′ as the detectable feature unchanged.

The above descriptions of the detailed embodiments are only to illustrate the principle and the effect of the present invention, and it is not to limit the scope of the present invention. Those skilled in the art can modify or change the embodiments without departing from the spirit and scope of the present invention. Accordingly, all equivalent modifications and variations completed by persons of ordinary skill in the art, without departing from the spirit and technical idea of the present invention, should fall within the scope of the present disclosure defined by the appended claims.

1. A delivery system including a position detecting unit, comprising:
a housing;
a reservoir supported by the housing;
a piston displaceable along the longitudinal axis in the reservoir, wherein the piston includes a detectable feature;
a lead screw directly connected with the piston;
at least two or more position sensors, wherein the position sensors are arranged within the longitudinal distance of the reservoir composing a sensor array, configured to sense a change of the magnetic field or the electric field caused by the displacement of the detectable feature and generate a signal. Based on the signal the position of the piston can be determined using algorithms, and any displacement abnormalities of the piston can be sensed from which infusion abnormalities can be deduced.

2. The delivery system including a position detecting unit according to claim 1, wherein,
the sensor array is a linear array of sensors with every sensor detecting the displacement of the piston for a specific range, configured to realize segmented detections of the detectable feature.

3. The delivery system including a position detecting unit according to claim 2, wherein,
the position sensors are arranged on the outer wall of the reservoir in the same housing with the reservoir.

4. The delivery system including a position detecting unit according to claim 2, wherein,
the position sensors are arranged adjacent to the reservoir in the other housing which can be connected with the housing supporting the reservoir.

5. The delivery system including a position detecting unit according to claim 2, wherein,
the number of the position sensors in the linear sensor array is 5 to 10, and the position sensors can realize joint detections with a specific algorithm.

6. The delivery system including a position detecting unit according to claim 2, wherein,
the position sensors are magnetic sensors.

7. The delivery system including a position detecting unit according to claim 6, wherein,
the detectable feature is the piston itself made of magnetic plastic.

8. The delivery system including a position detecting unit according to claim 6, wherein,
the detectable feature is a magnet foil which is mounted in a recess set on the piston.

9. The delivery system including a position detecting unit according to claim 6, wherein,
the detectable feature is a magnetic silicone sealing ring set in the circumferential direction on the piston.

10. The delivery system including a position detecting unit according to claim 6, wherein,
The detectable feature is a magnetic rubber sealing ring set in the circumferential direction on the piston.

11. The delivery system including a position detecting unit according to claim 2, wherein,
the detectable feature is a metal key which is mounted in a recess set on the piston.

12. The delivery system including a position detecting unit according to claim 11, wherein,
the position sensors are capacitive sensors.

13. The delivery system including a position detecting unit according to claim 11, wherein,
the position sensors are inductive sensors.

14. The delivery system including a position detecting unit according to claim 11, wherein,
the position sensors are eddy-current sensors.
